(19) United States
(12) Patent Application Publication
(10) Pub. No.: US 2025/0312248 A1
(43) Pub. Date: Oct. 9, 2025

(54) Oil-in-water emulsion comprising a semiochemical composition, suitable for administration in the form of a mist

(71) Applicant: SIGNS, APT (FR)

(72) Inventors: Maréva REBOUL, APT (FR); Mélinda TEP, APT (FR); Patrick PAGEAT, APT (FR)

(21) Appl. No.: 19/036,201

(22) Filed: Jan. 24, 2025

(30) Foreign Application Priority Data
Apr. 5, 2024 (FR) 2403566

(51) Int. Cl. A61K 8/06; A61Q 13/00

(52) U.S. Cl. CPC A61K 8/062; A61Q 13/00; A61K 2800/74; A61K 2800/87

(57) ABSTRACT

The invention relates to the field of semiochemicals, and describes in particular a composition in the form of an oil-in-water emulsion comprising an aqueous phase, and an oily phase comprising a semiochemical composition, in particular suitable for administration in the form of a mist, as well as its preparation process. The invention also relates to the non-therapeutic use of the composition according to the invention for administration, in particular by spraying using a mister, on the skin of an animal or a human, or on the skin of farm animals treated as a group, or on an element of the environment such as textiles and/or objects and/or walls, or by spraying into the air, in particular close to the nose of an animal or a human.

### TECHNICAL FIELD OF THE INVENTION

The invention relates to the field of semiochemicals, and describes in particular a composition in the form of an oil-in-water emulsion comprising an aqueous phase, and an oily phase comprising a semiochemical composition, in particular suitable for administration in the form of a mist, as well as its preparation process. The invention also relates to the non-therapeutic use of the composition according to the invention for administration, in particular by spraying using a mister, on the skin of an animal or a human, or on the skin of farm animals treated as a group, or on an element of the environment such as textiles and/or objects and/or walls, or by spraying into the air, in particular close to the nose of an animal or a human.

### PRIOR STATE OF THE ART

Semiochemicals according to the invention correspond to mixtures of several chemical compounds, described for the first time in particular by Silverstein, R. M. (1977). Semiochemicals are chemical substances released by a plant or animal that provoke a non-therapeutic behavioural or physiological response in another organism. When the semiochemical affects an individual of the same species (“intra-specific semiochemical product”), it is called a pheromone (Wyatt T. D., 2003). There are several types of pheromones that enable different types of behavioural interaction or communication, such as soothing pheromones, sex pheromones, territorial marking pheromones and warning pheromones. When the semiochemical affects an individual of a different species (“interspecific semiochemical”), it is called an allelochemical substance. Among allelochemical substances, an allomone is an allelochemical that triggers a reaction in the target species, this reaction being favourable to the species that releases the allomone (the opposite is a kairomone).

Since 1995, specialties whose active ingredients are semiochemical agents have been marketed. Some of their uses require the product to be deposited on the animal's skin, for example to obtain the desired effects on an animal in an open environment or to protect it from pests such as parasites or biting insects, or on human skin. The physico-chemical constraints of semiochemical components, and in particular their volatility and insolubility in water, have led to the use of excipients such as ethoxydiglycol in marketed specialties. The use of this type of excipient is now increasingly restricted by current regulations, but also by the general public, tending to reject them because of the way they are synthesised and their potential direct and environmental toxicity. It therefore appeared necessary to develop aqueous formulations of semiochemical compounds.

### DISCLOSURE OF THE INVENTION

The aim of the invention is to develop a composition based on semiochemical products (also referred to as compounds, active ingredients or agents) in a new galenic form, preferably non-toxic and environmentally friendly, in particular in the form of an oil-in-water (O/W) emulsion, preferably in the form of a stable and homogeneous oil-in-water (O/W) emulsion, for administration, in particular by spraying using a misting device, on the skin of an animal, in particular a mammal, or of a human, or on the skin of farm animals treated as a group, or on an element of the environment such as textiles and/or objects and/or walls, or by spraying into the air, in particular close to the nose of an animal or a human, in particular to reduce and/or avoid obtaining a wetting effect or stains; or to reach the vomeronasal and olfactory mucosa more quickly. Another aim of the invention is to develop a composition based on semiochemical products that has been positively evaluated in acceptability tests on target animals.

### SUMMARY OF THE INVENTION

The invention is defined by the independent claims. The dependent claims define particular or preferred embodiments of the invention.

According to a first aspect, the invention relates to a composition in the form of an oil-in-water (O/W) emulsion (referred to as an O/W composition or composition according to the invention), suitable in particular for the release of semiochemical compounds, comprising:
a) an aqueous phase comprising water and at least one humectant chosen from polyols and sugars,
b) an oily phase comprising at least one emollient agent, at least one emulsifying agent, and a semiochemical composition,
wherein said semiochemical composition comprises:
   methyl oleate and methyl palmitate, preferably methyl linoleate, methyl oleate and methyl palmitate; or
   3-cyclopentyl propionic acid and 3-cyclohexyl propionic acid; or
   bis(2-ethylhexyl) adipate and 1-isopropyl-2,2-dimethyltrimethylene diisobutyrate.

According to a second aspect, the invention relates to a process for preparing a composition in the form of an oil-in-water (O/W) emulsion according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent, said semiochemical composition and optionally said at least one additive are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, a perfume is added;
f) the composition is recovered in the form of an oil-in-water (O/W) emulsion obtained in step c), d) or e).

According to a third aspect, the invention relates to the non-therapeutic use of a composition according to the invention for administration, in particular by spraying with the aid of a mister, on the skin of an animal or a human, or on the skin of farm animals treated as a group, or on an element of the environment such as textiles and/or objects and/or walls, or by spraying into the air, in particular close to the nose of an animal or a human.

### DETAILED DESCRIPTION OF THE INVENTION

The invention is now described in more detail and in a non-limiting way in the following description. Unless otherwise stated, all percentages relating to quantities are percentages by mass. Any reference to ranges of values in the description and/or claims implies, unless otherwise stated, that the limits of the ranges are included.

The invention relates to a composition in the form of an oil-in-water (O/W) emulsion comprising:
a) an aqueous phase comprising water and at least one humectant chosen from polyols and sugars,
b) an oily phase comprising at least one emollient agent, at least one emulsifying agent, and a semiochemical composition,
wherein said semiochemical composition comprises:
   methyl oleate and methyl palmitate, preferably methyl linoleate, methyl oleate and methyl palmitate; or
   3-cyclopentyl propionic acid and 3-cyclohexyl propionic acid; or
   bis(2-ethylhexyl) adipate and 1-isopropyl-2,2-dimethyltrimethylene diisobutyrate.

In a particular embodiment of the invention, said semiochemical composition comprises:
methyl oleate, methyl linoleate, methyl behenate, methyl palmitate, methyl myristate and methyl laurate; or
methyl linoleate, methyl oleate, methyl palmitate, methyl myristate, methyl laurate and methyl decanoate; or
methyl linoleate, methyl oleate, methyl palmitate, methyl stearate, methyl myristate, methyl pentadecanoate and methyl laurate; or
methyl linoleate, methyl oleate, methyl palmitate, 2-(2-butoxyethoxy) ethanol, dimethyl glutarate, dimethyl adipate, dimethyl succinate and dodecane; or
methyl oleate, methyl palmitate, methyl linoleate, methyl stearate, methyl myristate and methyl pentadecanoate; or
methyl oleate, methyl linoleate, methyl palmitate, squalene, methyl myristate and methyl pentadecanoate; or
methyl oleate, methyl palmitate, dimethyl pimelate and dimethyl azelate; or
methyl laurate, methyl myristate, methyl palmitate, methyl stearate, methyl oleate, methyl linoleate, dimethyl pimelate and dimethyl azelate; or
3-cyclopentyl propionic acid and 3-cyclohexyl propionic acid; or
bis(2-ethylhexyl) adipate and 1-isopropyl-2,2-dimethyltrimethylene diisobutyrate; or
methyl oleate, methyl palmitate, methyl linoleate, dioxolane, methyl laurate and methyl myristate.

In a preferred embodiment of the invention, said semiochemical composition comprises:
from 25% to 35% by weight of methyl oleate, from 20% to 25% by weight of methyl linoleate, from 20% to 25% by weight of methyl behenate, from 20% to 25% by weight of methyl palmitate, from 3% to 6% by weight of methyl myristate and from 2% to 4% by weight of methyl laurate; or
from 30% to 40% by weight of methyl linoleate, from 25% to 35% by weight of methyl oleate, from 15% to 25% by weight of methyl palmitate, from 5% to 9% by weight of methyl myristate, from 5% to 7% by weight of methyl laurate and from 1% to 3% by weight of methyl decanoate; or
from 25% to 35% by weight of methyl linoleate, from 25% to 35% by weight of methyl oleate, from 15% to 25% by weight of methyl palmitate, from 10% to 15% by weight of methyl stearate, from 2% to 7% by weight of methyl myristate, from 2% to 6% by weight of methyl pentadecanoate and from 1% to 3% by weight of methyl laurate; or
from 30% to 40% by weight of methyl linoleate, from 25% to 35% by weight of methyl oleate, from 15% to 25% by weight of methyl palmitate, from 5% to 15% by weight of 2-(2-butoxyethoxy) ethanol, from 2% to 6% by weight of dimethyl glutarate, from 1% to 3% by weight of dimethyl adipate, from 1% to 3% by weight of dimethyl succinate and from 1% to 3% by weight of dodecane; or
from 25% to 35% by weight of methyl oleate, from 25% to 35% by weight of methyl palmitate, from 25% to 35% by weight of methyl linoleate, from 3% to 7% by weight of methyl stearate, from 3% to 5% by weight of methyl myristate and from 0.5% to 2% by weight of methyl pentadecanoate; or
from 30% to 40% by weight of methyl oleate, from 20% to 26% by weight of methyl linoleate, from 20% to 26% by weight of methyl palmitate, from 6% to 10% by weight of squalene, from 2% to 6% by weight of methyl myristate and from 3% to 5% by weight of methyl pentadecanoate; or
from 62% to 70% by weight of methyl oleate, from 12% to 16% by weight of methyl palmitate, from 7% to 13% by weight of dimethyl pimelate and from 6% to 11% by weight of dimethyl azelate; or
from 2% to 6% by weight of methyl laurate, from 2% to 6% by weight of methyl myristate, from 11% to 15% by weight of methyl palmitate, from 2% to 6% by weight of methyl stearate, from 42% to 50% by weight of methyl oleate, from 4% to 8% by weight of methyl linoleate, from 7% to 13% by weight of dimethyl pimelate and from 6% to 11% by weight of dimethyl azelate; or
from 40% to 60% by weight of 3-cyclopentyl propionic acid and from 40% to 60% by weight of 3-cyclohexyl propionic acid; or
from 40% to 60% by weight of bis(2-ethylhexyl) adipate and from 40% to 60% by weight of 1-isopropyl-2,2-dimethyltrimethylene diisobutyrate, or
from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate; the total sum of the compounds present in said semiochemical composition being equal to 100%.

### Dosage Form and Administration

The composition according to the invention is in the form of an oil-in-water (O/W) emulsion, in particular an oil-in-water emulsion which is stable and has a homogeneous appearance, particularly to the naked eye.

In the context of the invention, the term “emulsion” refers to a mixture of two immiscible liquid substances, such as oil and water. In an oil-in-water (O/W) emulsion, the oil is dispersed in the water in the form of droplets, particularly small droplets.

In the context of the invention, the term “stable emulsion” refers to an emulsion showing no signs of instability (phase separation, increase in droplet size, etc.), in particular an emulsion which does not release water or oil and which remains in its initial form over time and under different temperature conditions, for example during a 3 months storage at a temperature of between 5° C. and 40° C.

In the context of the invention, the term “homogeneous emulsion” refers to an emulsion whose surface is smooth and uniform to the naked eye.

In particular, the composition can be administered by means of a spraying device, in particular by means of a mister, in the form of a fluid, opalescent, transparent or translucent mixture, in particular in the form of a mist.

In the context of the invention, the term “mist” refers to an aqueous dispersion in droplets comparable to a cloud of droplets or light fog.

In the context of the invention, the term “fluid mixture” refers to a low-viscosity mixture with the ability to flow under its own weight, unlike a solid composition.

In the context of the invention, the term “opalescent” refers to a milky and iridescent colour, a reflection reminiscent of opal.

In the context of the invention, the term “transparent” means that the mixture allows light to pass through and objects behind it to be seen clearly.

In the context of the invention, the term “translucent” means that the mixture allows light to pass through but does not give a clear view of the objects behind it, which remain blurred.

The O/W composition according to the invention can be applied to small and/or large areas of the body (e.g., the skin of an animal or human) and/or material (e.g., an element of the environment such as clothing and/or objects and/or walls).

Preferably, the O/W composition according to the invention is administered in the form of a mist using a mister.

Non-limiting examples of misters are well known to those skilled in the art.

Preferably, the mist, in particular the droplets of this mist, is opalescent, transparent or translucent, preferably transparent.

### Aqueous Phase

The composition according to the invention comprises an aqueous phase, in particular a continuous aqueous phase (also known as the external phase).

The aqueous phase of the composition according to the invention comprises water, preferably demineralised water or purified water, and optionally one or more water-miscible or at least partly water-miscible compounds, for example a skin care agent such as ALOE VERA, a hydrolat, urea or an antistatic agent.

In the context of the invention, the term “skin care agent” refers to an agent for maintaining the skin in good condition, e.g., healthy-looking.

In the context of the invention, the term “antistatic agent” refers to an agent that reduces static electricity by neutralising the electrical charge on a surface.

In a particular embodiment of the invention, the composition according to the invention comprises from 60% to 95% by weight of water, preferably from 77% to 87% by weight of water, relative to the total weight of the composition.

In a preferred embodiment of the invention, the composition according to the invention comprises from 62% to 91% by weight of water, preferably from 77% to 87% by weight of water, even more preferably 82.5% or 83.9% or 85.7% by weight of water, relative to the total weight of the composition.

The aqueous phase of the composition according to the invention may comprise water and at least one humectant chosen from polyols and sugars.

### Humectant

According to a particular embodiment, the aqueous phase of the composition according to the invention also comprises at least one humectant chosen from polyols and sugars. In particular, the aqueous phase of the composition according to the invention also comprises a single humectant chosen from polyols or sugars, preferably polyols. Said at least one humectant is notably soluble in the aqueous phase.

In the context of the invention, the term “humectant” (also called moisturising agent) refers to a compound or mixture of compounds that retain water thanks to their hygroscopic properties.

In the context of the invention, the term “polyol” refers to any organic compound comprising at least two free hydroxyl groups (—OH), such as glycols.

In the context of the invention, the term “sugar” refers to simple carbohydrates, in particular any organic molecule consisting of one ose (monosaccharide) or two oses (disaccharide).

In a particular embodiment of the invention, said at least one humectant is chosen from polyols selected from the group consisting of glycerol (also called glycerine), propylene glycol, polyethylene glycols (PEG), xylitol, isomalt, maltitol, mannitol, polydextrose, sorbitol, isomaltitol, lactitol, and mixtures thereof; and/or said at least one humectant is chosen from sugars selected from the group consisting of fructose, glucose, lactose and mixtures thereof.

In a preferred embodiment of the invention, said at least one humectant is a polyol, in particular glycerol.

Said at least one humectant, i.e. one or more humectant(s), preferably one humectant, may be present in the composition in a content ranging from 4% to 12% or 4.5% to 12% by weight, preferably from 4% to 6% by weight, relative to the total weight of the composition.

Preferably, said at least one humectant is glycerol and is present in the composition in an amount ranging from 4.5% to 12% by weight, preferably 5% by weight, relative to the total weight of the composition.

In a particular embodiment of the invention, the aqueous phase of the composition comprises water in a content ranging from 62% to 91% by weight, preferably from 77% to 87% by weight, even more preferably 82.5% or 83.9% or 85.7% by weight, and glycerol in a content ranging from 4.5% to 12% by weight, preferably 5% by weight, all the percentages being given relative to the total weight of the composition.

### Oily Phase

The composition according to the invention comprises an oily phase, in particular a dispersed oily phase (also called discontinuous or internal phase).

The oily phase of the composition according to the invention may comprise at least one emollient agent, at least one emulsifying agent and a semiochemical composition. In particular, said at least one emollient agent, said at least one emulsifying agent and said semiochemical composition are notably soluble in the oily phase.

The oily phase may be present in an amount ranging from 3% to 45% by weight, preferably from 10% to 25% by weight, relative to the total weight of the composition.

### Emollient Agent

According to a particular embodiment, the oily phase of the composition according to the invention also comprises at least one emollient agent. In particular, the oily phase of the composition according to the invention also comprises a single emollient agent. Said at least one emollient agent is notably soluble in the oily phase.

In the context of the invention, the term “emollient agent” refers to an agent for enhancing the softness and suppleness of the skin during topical, in particular cutaneous, administration of the composition according to the invention.

In a particular embodiment of the invention, said at least one emollient agent is selected from the group consisting of hydrogenated vegetable oils (vegetable oils comprising triglycerides of unsaturated fatty acids, for example hydrogenated olive oil, hydrogenated rapeseed oil, hydrogenated sunflower oil, hydrogenated sesame oil, hydrogenated linseed oil, hydrogenated hemp oil, hydrogenated walnut oil, hydrogenated hazelnut oil, hydrogenated coconut oil, hydrogenated argan oil, hydrogenated jojoba oil, hydrogenated sweet almond oil, hydrogenated shea butter, hydrogenated castor oil, hydrogenated avocado oil, hydrogenated apricot kernel oil, hydrogenated macadamia oil, . . . ), silicone oils (e.g., dimethicone, . . . ), mineral oils and waxes (e.g., Parrafine, . . . ), fatty alcohols (e.g., arachidic alcohol, batylic alcohol, behenyl alcohol, C₁₂-C₁₃ alcohols, cetearyl alcohol, cetyl alcohol, coconut alcohol, isocetyl alcohol, octyldodecanol, palm alcohol, . . . ), fatty acids and fatty acid esters (e.g., batyl stearate, cetearyl behenate, cetearyl isononanoate, cetyl esters, coconut acid, ethyl linoleate, ethyl linolenate, ethyl olivate, ethylhexyl cocoate, ethylhexyl myristate, glyceryl caprylate, glyceryl laurate/oleate, glyceryl ricinoleate, isocetyl salicylate, isostearyl alcohol, linoleic acid, linolenic acid, oleic acid, palmitic acid, palmitic acid, wheat germ acid, . . . ), glyceride derivatives (e.g., triglycerides of capric acid and caprylic acid, cocoglycerides, palm glycerides, . . . ) and glycerol derivatives (e.g., glycerol trioctanoate: Tricaprylin, Glycerol tristearate: Tristearin . . . ), preferably glyceride derivatives such as fatty acid triglycerides.

Said at least one emollient agent, i.e., one or more emollient agent(s), is/are present in an amount ranging from 0.5% to 8% by weight, preferably from 1% to 3% by weight, relative to the total weight of the composition.

Preferably, said at least one emollient agent is a glyceride derivative, in particular a fatty acid triglyceride, in particular a triglyceride of caprylic acid and capric acid such as that marketed in particular by BASF under the name Myritol 318®, and is present in the composition in an amount ranging from 0.5% to 8% by weight, preferably 2% by weight, relative to the total weight of the composition.

In a particular embodiment of the invention, the aqueous phase of the composition comprises water in an amount ranging from 62% to 91% by weight, preferably from 77% to 87% by weight, even more preferably 82.5% or 83.9% or 85.7% by weight, and glycerol in an amount ranging from 4.5% to 12% by weight, preferably 5% by weight, and the oil phase of the composition comprises a triglyceride of caprylic acid and capric acid such as Myritol 318® in an amount ranging from 0.5% to 8% by weight, preferably 2% by weight, all the percentages being given relative to the total weight of the composition.

### Emulsifying Agent

The oily phase of the composition according to the invention also comprises at least one emulsifying agent, preferably one or more emulsifying agent(s). Said at least one emulsifying agent is notably soluble in the oily phase.

In the context of the invention, the term “emulsifying agent” (also called emulsifier) refers to a small molecule composed of a hydrophilic head soluble in the aqueous phase and a lipophilic tail with an affinity for the oily phase. Said at least one emulsifying agent enables the formation of an interface film around the droplets in the dispersed phase, thus forming the emulsion and providing stability to the formed emulsion.

Said at least one emulsifying agent may be chosen from non-ionic or ionic (anionic, cationic) surfactants, preferably non-ionic surfactants.

In the context of the invention, the term “surfactant” refers to a compound which reduces surface tension and promotes uniform distribution of the composition according to the invention when used.

In a particular embodiment of the invention, said at least one emulsifying agent is an ionic surfactant, preferably an anionic surfactant chosen from sulphated castor oil, sodium dodecyl sulphate (SDS), sodium coco sulphate (INCI name: Sodium coco sulfate (SCS)), sodium cocoyl isethionate (INCI name: Sodium cocoyl isethionate (SCI)), sodium lauryl sulphoacetate (INCI name: Sodium lauryl sulfoacetate (SLSA)), sodium laureth sulphate (INCI name: Sodium laureth sulfate (SLES)) and sodium lauroyl sarcosinate (INCI name: Sodium Lauroyl Sarcosinate), or a cationic surfactant chosen from cetearyl alcohol (INCI name: Cetearyl Alcohol), behentrimonium methosulphate (INCI name: Behentrimonium Methosulfate (BTMS)), brassica alcohol (and) brassicycle valinate esylate (INCI name: Brassica Alcohol (and) Brassicyl Valinate Esylate), benzalkonium chloride (BAC), didecyldimethylammonium chloride (DDAC), brassicamidopropyl dimethylamine (INCI name: Brassicamidopropyl dimethylamine), Polyquaternium-7, Polyquaternium-10, Polyquaternium-22 and Polyquaternium-37.

In a preferred embodiment of the invention, said at least one emulsifying agent is a non-ionic surfactant, preferably a non-ionic surfactant having an HLB of between 8 and 20 at 25° C., and is selected from the group consisting of in particular polyoxyethylene sorbitan fatty acid esters, polyol fatty acid esters and ethoxylated, preferably hydrogenated and ethoxylated, vegetable oils.

Examples of polyoxyethylene sorbitan fatty acid esters (also called esters of fatty acid and polyoxyethylene sorbitan; ethoxylated sorbitan, sorbitan esters) include in particular esters of C₈-C₂₄, preferably C₁₆-C₂₂, fatty acids and of polyoxyethylene sorbitan which may comprise from 1 to 150 oxyethylenated groups, such as polysorbate 20 in particular marketed under the name Tween 20® by CRODA, polysorbate 21 in particular marketed under the name Tween 21® by CRODA, polysorbate 40 in particular marketed under the name Tween 40® by CRODA, polysorbate 60 in particular marketed under the name Tween 60® by CRODA, polysorbate 61 in particular marketed under the name Tween 61® by CRODA, polysorbate 65 in particular marketed under the name Tween 65® by CRODA, polysorbate 80 in particular marketed under the name Tween80® by CRODA, polysorbate 81 in particular marketed under the name Tween 81@ by CRODA and polysorbate 85 in particular marketed under the name Tween 85® by CRODA, preferably polysorbate 80 or polysorbate 20.

Examples of fatty acid esters of polyols, in particular polyglycerols, include polyglyceryl-3 beeswax, polyglyceryl-6 distearate, polyglyceryl-4 caprate, or polyglyceryl-3 distearate in particular marketed by BASF under the name Cremophor GS 32®, preferably Cremophor GS 32®.

Examples of ethoxylated vegetable oils, preferably hydrogenated and ethoxylated vegetable oils, include in particular ethoxylated and hydrogenated castor oil, in particular PEG-35 or PEG-40 hydrogenated castor oil, preferably PEG-40 hydrogenated castor oil in particular marketed by BASF under the name Eumulgin CO 40®, preferably Eumulgin CO 40®.

In a particular embodiment of the invention, said at least one emulsifying agent, i.e., one or more emulsifying agent(s), is/are present in the composition in an amount ranging from 1% to 25% by weight, preferably from 5% to 7% by weight, relative to the total weight of the composition.

In a preferred embodiment of the invention, the composition according to the invention comprises several emulsifying agents, in particular a mixture of emulsifying agents consisting of polyoxyethylene sorbitan fatty acid esters, polyol fatty acid esters and ethoxylated, preferably hydrogenated and ethoxylated, vegetable oils.

Preferably, said emulsifying agents are polyglyceryl-3 distearate marketed in particular by BASF under the name Cremophor GS 32®, PEG-40 hydrogenated castor oil marketed in particular by BASF under the name Eumulgin CO 40®, polyoxyethylene sorbitan fatty acid esters such as polysorbate 80 marketed in particular under the name Tween 80® by CRODA, or polysorbate 20 marketed in particular under the name Tween 20® by CRODA, and said emulsifying agents are present in the composition in an amount ranging from 1% to 25% by weight, preferably from 5% to 7% by weight, even more preferably of 6% by weight, relative to the total weight of the composition. In particular, Cremophor GS 32® is present in the composition in an amount ranging from 0.6% to 8.5% by weight, preferably 2% by weight, relative to the total weight of the composition; and/or Eumulgin CO 40® is present in the composition in an amount ranging from 0.6% to 8.5% by weight, preferably 2% by weight, relative to the total weight of the composition; and/or polysorbate 80 is present in the composition in an amount ranging from 0.5% to 7.5% by weight, preferably 2% by weight, relative to the total weight of the composition; and/or polysorbate 20 is present in the composition in an amount ranging from 0.5% to 7.5% by weight, preferably 2% by weight, relative to the total weight of the composition.

In a particular embodiment of the invention, the aqueous phase of the composition comprises water in an amount ranging from 62% to 91% by weight, preferably from 77% to 87% by weight, even more preferably 82.5% or 83.9% or 85.7% by weight, and glycerol in an amount ranging from 4.5% to 12% by weight, preferably 5% by weight, and the oily phase of the composition comprises a triglyceride of caprylic acid and capric acid such as Myritol 318® in an amount ranging from 0.5% to 8% by weight, preferably 2% by weight, and a mixture of emulsifying agents consisting of Cremophor GS 32® in an amount ranging from 0.6% to 8.5% by weight, preferably 2% by weight, Eumulgin CO 40® in an amount ranging from 0.6% to 8.5% by weight, preferably 2% by weight, polysorbate 80 or polysorbate 20 in an amount ranging from 0.5% to 7.5% by weight, preferably 2% by weight, all the percentages being given relative to the total weight of the composition.

### Semiochemical Composition

The oily phase of the composition according to the invention also comprises a semiochemical composition as an active agent, as defined above. Said semiochemical composition is notably soluble in the oily phase described herein. Particular semiochemical compositions are described above and in the preferred embodiments and experimental examples that follow. The semiochemical compositions contained in the compositions according to the invention are in particular intended for non-therapeutic use, for example use for their behavioural or protective effect against pests (parasites, insects, etc.) on the animals to which they are administered or on animals interacting with them, or for their well-being effect on humans.

The semiochemical composition may be present in the composition in the form of an oil-in-water emulsion in an amount ranging from 1% to 4% by weight, preferably from 1.5% to 2.5% by weight, even more preferably from 1.98% or 2% by weight of said semiochemical composition, relative to the total weight of the composition according to the invention.

### Additives

The composition according to the invention may further comprise at least one additive provided that it does not interfere with the desired properties of the composition, in particular with its property of being an O/W emulsion.

In a particular embodiment of the invention, said at least one additive is selected from the group consisting of a preservative, in particular an antimicrobial agent or an antioxidant agent, a perfume, a masking agent, and mixtures thereof.

In the context of the invention, the term “preservative” refers to an agent that protects the composition according to the invention from changes in its physical or chemical state, its colour or its odour, or protects it from microbial contamination or degradation, for example by inhibiting the development of micro-organisms during the time the composition according to the invention is stored or used. Such an agent must be compatible with use of the composition according to the invention in contact with an animal or a human, and is advantageously non-irritating to the skin and eyes.

Examples of preservatives are known to the skilled person and include benzyl alcohol, didecyldimonium chloride, caprylyl glycol, phenylpropanol, sodium benzoate, phenyl acetate, sorbic acid, dehydroacetic acid, potassium sorbate, phenoxyethanol, cetrimonium bromide, triclosan, parabens and thiazolinones.

In the context of the invention, the term “antimicrobial agent” therefore refers to a preservative that slows down the growth of micro-organisms (bacteria, fungi, yeasts, moulds) and combats the development of microbes in the composition according to the invention during its storage and/or when it is used.

Examples of antimicrobial agents are known to those skilled in the art and include glycerine/phenyl alcohol/caprylhydroxamic acid, marketed in particular under the name Aromastat® by INOLEX.

The composition according to the invention may comprise from 0% to 4% by weight, preferably from 0.2% to 3.2% by weight, even more preferably 1% by weight of preservative, in particular antimicrobial agent, relative to the total weight of the composition according to the invention.

In the context of the invention, the term “antioxidant agent” refers to a preservative capable of inhibiting reactions promoted by oxygen, thereby preventing oxidation.

Examples of antioxidant agents are known to those skilled in the art and include in particular compounds of the cinnamic acid family and their derivatives, such as pentaerythrityl tetra-di-t-butyl hydroxyhydrocinnamate, marketed in particular under the name Tinogard TT® by BASF, vitamin A (retinol), vitamin C (ascorbic acid) and derivatives (ascorbate), vitamin E (tocopherol) and derivatives (tocopheryl), preferably Tinogard TT®.

The composition according to the invention may comprise from 0% to 2% by weight, preferably from 0.02% to 0.3% by weight, even more preferably 0.1% by weight of antioxidant agent, relative to the total weight of the composition.

In the context of the invention, the term “perfume” or perfuming agent refers to any hydrophobic substance designating a pure substance or a mixture that is insoluble or sparingly soluble in water and capable of giving off a pleasant odour.

The composition according to the invention may comprise from 0% to 3% by weight, preferably from 0.2% to 2.5% by weight, even more preferably 1% by weight of perfume, relative to the total weight of the composition.

In the context of the invention, the term “masking agent” refers to an agent that reduces or inhibits the basic odour or taste of the product.

Examples of masking agents are known to the skilled person and include in particular beta sitosterol, citric acid, bisabolol and Calendula officinalis flower extract.

The composition according to the invention can comprise from 0% to 3% by weight, preferably from 0.2% to 2.5% by weight, even more preferably 1% by weight of a masking agent, relative to the total weight of the composition.

In a particular embodiment of the invention, said at least one additive is added to the oily phase, i.e., the oily phase further comprises at least one additive as defined above.

In another particular embodiment of the invention, said at least one additive, for example a perfume, is added after the formation of the oil-in-water emulsion, or after a step for adjusting the pH of the oil-in-water emulsion.

In a preferred embodiment of the invention, the oily phase also comprises at least one additive selected from the group consisting of a preservative, in particular an antimicrobial agent or an antioxidant agent, a perfume, a masking agent, and mixtures thereof, even more preferably at least one additive selected from the group consisting of a preservative, in particular an antimicrobial agent or an antioxidant agent, a masking agent, and mixtures thereof.

In another preferred embodiment of the invention, the composition according to the invention also comprises at least one additive such as a perfume. In particular, said perfume is added after the formation of the oil-in-water emulsion or after a step for adjusting the pH of the oil-in-water emulsion in order to avoid degradation of the perfume or loss of the perfume odour.

In a particular embodiment of the invention, the composition comprises from 60% to 95% by weight of water, preferably from 77% to 87% by weight of water, relative to the total weight of the composition, and/or comprises from 1% to 4% by weight, preferably from 1.5% to 2.5% by weight of said semiochemical composition, relative to the total weight of the composition.

In a preferred embodiment of the invention, the composition according to the invention comprises:
a) an aqueous phase comprising water and glycerol,
b) an oily phase comprising polyglyceryl-3 distearate (Cremophor GS 32® in particular marketed by BASF), PEG-40 hydrogenated castor oil (Eumulgin CO 40® in particular marketed by BASF), polyoxyethylene sorbitan fatty acid esters (polysorbate 80 in particular marketed under the name Tween 808 by CRODA or polysorbate 20 in particular marketed under the name Tween 20® by CRODA), triglycerides of caprylic acid and capric acid (Myritol 318® in particular marketed by BASF), Glycerine/phenyl alcohol/caprylhydroxamic acid (Aromastat® in particular marketed by INOLEX), pentaerythrityl tetra-di-t-butyl hydroxyhydrocinnamate (Tinogard TT® in particular marketed by BASF), an optional perfume, and a semiochemical composition,
the semiochemical composition being present in the composition in the form of an oil-in-water emulsion in an amount ranging from 1% to 4% by weight, preferably from 1.5% to 2.5% by weight, relative to the total weight of the composition according to the invention.

In an even more preferred embodiment of the invention, the composition according to the invention comprises:
a) an aqueous phase comprising water in an amount ranging from 62% to 91% by weight, preferably from 77% to 87% by weight, even more preferably 82.5% or 83.9% or 85.7% by weight, and glycerol in an amount of from 4.5% to 12% by weight, preferably 5% by weight,
b) an oily phase comprising polyglyceryl-3 distearate (Cremophor GS 32® marketed in particular by BASF) in an amount ranging from 0.6% to 8.5% by weight, preferably 2% by weight, PEG-40 hydrogenated castor oil (Eumulgin CO 40®, in particular marketed by BASF) in an amount ranging from 0.6% to 8.5% by weight, preferably 2% by weight, polyoxyethylene sorbitan fatty acid esters (polysorbate 80, marketed in particular under the name Tween 80® by CRODA or polysorbate 20, marketed in particular under the name Tween 20® by CRODA) in an amount ranging from 0.5% to 7.5% by weight, preferably 2% by weight, triglycerides of caprylic acid and capric acid (Myritol 318® in particular marketed by BASF) in an amount ranging from 0.5% to 8% by weight, preferably 2% by weight, Glycerine/phenyl alcohol/caprylhydroxamic acid (Aromastat®, in particular marketed by INOLEX) in an amount ranging from 0.2% to 3.2% by weight, preferably 1% by weight, pentaerythrityl tetra-di-t-butyl hydroxyhydrocinnamate (Tinogard TT®, in particular marketed by BASF) in an amount ranging from 0.02% to 0.3% by weight, preferably 0.1% by weight, an optional perfume in an amount ranging from 0.2% to 2.5% by weight, preferably 1% by weight, and a semiochemical composition,
the semiochemical composition being present in the composition in the form of an oil-in-water emulsion in an amount ranging from 1% to 4% by weight, preferably from 1.5% to 2.5% by weight, the amounts being expressed by weight relative to the total weight of the O/W composition according to the invention.

In a particular embodiment of the invention, the composition has a Brookfield viscosity of less than 8500 mPa·s, in particular a viscosity of between 1 and 50 cP or mPa·s, or a viscosity of between 3500 and 8000 cP or mPa·s, as measured at 25° C. The viscosity within the meaning of the present invention is a Brookfield viscosity measured at room temperature (25° C.), preferably after 24 hours, by means of a viscometer, for example a Brookfield digital viscometer (model LDVI Prime), mobile S 61 and rotation speed 0.6 RPM.

In a particular embodiment of the invention, the composition in the form of an oil-in-water (O/W) emulsion has a pH of between 5 and 8, preferably a pH of between 5.5 and 7.5, or a pH of between 6.0 and 7.5.

Preferably, the composition has a pH value compatible with the topical, in particular cutaneous, application of the composition to animal or human skin.

### Preparation Processes for the Composition of the Invention

The invention also relates to a first process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent, said semiochemical composition and optionally said at least one additive are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, a perfume is added;
f) the composition is recovered in the form of an oil-in-water (O/W) emulsion obtained in step c), d) or e).

The invention also relates to a second process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent and said semiochemical composition are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, said at least one additive is added;
f) the composition is recovered in the form of an oil-in-water (O/W) emulsion obtained in step c), d) or e).

Preferably, the invention relates to the first process for preparing a composition according to the invention.

The definitions and/or particular/preferred embodiments mentioned in the following paragraphs are applicable to both the first and second processes for preparing a composition according to the invention.

In the context of the invention, the term “pasting” means that before adding the water in step a) of the process, the at least one gelling agent is dispersed in the at least one humectant, in order to obtain a smooth and homogeneous paste. This pasting (or mashing) step, known to those skilled in the art, separates the particles and prevents the formation of lumps.

In a particular embodiment of the first or second process according to the invention, the pasting step a) is carried out at room temperature; and/or
the homogeneous aqueous phase obtained in step a) is kept under stirring at a temperature of between 65° C. and 75° C., preferably at a temperature of 70° C.; and/or
the homogeneous oily phase obtained in step b) is kept under stirring at a temperature of between 65° C. and 75° C., preferably at a temperature of 70° C.; and/or
step c) is carried out by mixing the oily phase from step b) with the aqueous phase from step a) at a temperature of between 65° C. and 75° C., preferably at a temperature of 70° C., and the emulsion is cooled to room temperature; and/or
step d) is carried out by adding a basic solution, for example a NaOH solution, in particular a 10% NaOH solution, in order to increase the pH of the emulsion obtained in step c), preferably in order to obtain a pH of between 5 and 8, preferably a pH of between 5.5 and 7.5, or a pH of between 6.0 and 7.5.

In a particular embodiment of the process according to the invention, the aqueous phase obtained in step a) is in the form of a homogeneous mixture.

In a particular embodiment of the process according to the invention, the oily phase obtained in step b) is in the form of a homogeneous mixture.

In the context of the invention, the term “homogeneous mixture” refers to a mixture in which the individual constituents cannot be distinguished by the naked eye.

In a particular embodiment of the process according to the invention, the oil-in-water (O/W) emulsion obtained in step c) is a stable and homogeneous emulsion as defined above.

In a particular embodiment of the process according to the invention, step d) is mandatory for the preparation of a composition in the form of an oil-in-water (O/W) emulsion, comprising a semiochemical composition comprising from 40% to 60% of 3-cyclopentyl propionic acid and from 40% to 60% of 3-cyclohexyl propionic acid; preferably 50% of 3-cyclopentyl propionic acid and 50% of 3-cyclohexyl propionic acid (SC9).

The invention relates in particular to a process for preparing a composition according to the invention, in particular a first or second process for preparing a composition according to the invention as described above, in which the compounds of the aqueous and oily phases are as described above.

It is also the object of the invention to use a composition consisting of an aqueous phase and a composition consisting of an oily phase as described above and exemplified below, to produce a composition according to the invention comprising, as active agent, a semiochemical composition (associated with the oily phase), in the form of an oil-in-water (O/W) emulsion, in particular an oil-in-water emulsion which is stable and has a homogeneous appearance. In particular, the composition is suitable for use with a spraying device, such as a mister.

### Non-Therapeutic Use According to the Invention

According to another aspect, the invention relates to a non-therapeutic use of a composition according to the invention. According to this use, the composition according to the invention is used to obtain the semiochemical effect(s) expected from the semiochemical composition it contains. Thus, a composition according to the invention is used to improve the management of the behaviour or interactions of target animals or their well-being, by transmitting information or signals vectored by semiochemical agents, or to improve the well-being of humans.

In particular, the invention relates to a non-therapeutic use of a composition in the form of an oil-in-water (O/W) emulsion according to the invention, for administration, in particular by spraying with the aid of a mister, on the skin of an animal or a human, or on the skin of farm animals treated as a group, or on textiles and/or objects and/or walls, or by spraying into the air, in particular close to the nose of an animal or a human.

One use of the composition according to the invention is, for example, as a tool or means of improving animal husbandry. One use of the composition according to the invention is, for example, as an adaptive tool or means for the animal in relation to its environment.

Non-therapeutic use of the composition according to the invention comprises the topical, in particular cutaneous, administration of said composition to an animal, in particular a mammal, or to a human, or to animals treated as a group.

The non-therapeutic use of the composition according to the invention comprises the administration, in particular by spraying using a misting device, onto the skin of an animal or human, or onto the skin of farm animals treated as a group, or onto an element of the environment such as textiles and/or objects and/or walls, or by spraying into the air, in particular close to the nose of an animal or human, in particular to reduce and/or avoid obtaining a wetting effect or stains; or to reach the vomeronasal and olfactory mucosa more quickly.

In a particular embodiment of the invention, the composition is administered topically, in particular cutaneously, to the skin of an animal or human, preferably to the skin of an animal.

In a particular embodiment according to the invention, the animal is a domestic animal or a farm animal, in particular a mammal, preferably the animal is selected from the group consisting of pets such as rabbits, cats, dogs, birds or from the group of farm animals such as horses or production animals such as pigs, cattle, sheep, poultry, for example hens.

In a particular embodiment according to the invention, the composition comprises a semiochemical composition, in particular comprising a bovine soothing pheromone, comprising from 25% to 35% by weight of methyl oleate, from 20% to 25% by weight of methyl linoleate, from 20% to 25% by weight of methyl behenate, from 20% to 25% by weight of methyl palmitate, from 3% to 6% by weight of methyl myristate and from 2% to 4% by weight of methyl laurate; and relates to the non-therapeutic use of said composition for administration, in particular by spraying using a mister, in particular topical administration, in particular cutaneous administration to cattle or cattle treated as a group, in particular on the skin of a cattle or cattle, in particular to promote soothing states in the cattle.

In a particular embodiment according to the invention, the composition comprises a semiochemical composition, in particular comprising a soothing pheromone for pigs, comprising from 30% to 40% by weight of methyl linoleate, from 25% to 35% by weight of methyl oleate, from 15% to 25% by weight of methyl palmitate, from 5% to 9% by weight of methyl myristate, from 5% to 7% by weight of methyl laurate and from 1% to 3% by weight of methyl decanoate; and relates to the non-therapeutic use of said composition for administration, in particular by spraying using a mister, in particular topical administration, in particular cutaneous administration to pigs or groups of pigs, in particular on the skin of a pig or pigs, in particular to promote soothing states in pigs.

In a particular embodiment according to the invention, the composition comprises a semiochemical composition, in particular comprising a dog-soothing pheromone, comprising from 25% to 35% by weight of methyl linoleate, from 25% to 35% by weight of methyl oleate, from 15% to 25% by weight of methyl palmitate, from 10% to 15% by weight of methyl stearate, from 2% to 7% by weight of methyl myristate, from 2% to 6% by weight of methyl pentadecanoate and from 1% to 3% by weight of methyl laurate; and relates to the non-therapeutic use of said composition for administration, in particular by spraying using a mister, in particular topical administration, in particular cutaneous administration to dogs, in particular on the skin of a dog, or administration on an element of the environment such as textiles and/or objects and/or walls, in particular to facilitate adaptation and prevent stress induced by encounters with unknown dogs, humans or animals, and/or prevent stress reactions induced by external factors such as fireworks, storms, violent noises or unusual environments.

In a particular embodiment according to the invention, the composition comprises a semiochemical composition, in particular comprising a rabbit-soothing pheromone, comprising from 30% to 40% by weight of methyl linoleate, from 25% to 35% by weight of methyl oleate, from 15% to 25% by weight of methyl palmitate, from 5% to 15% by weight of 2-(2-butoxyethoxy) ethanol, from 2% to 6% by weight of dimethyl glutarate, from 1% to 3% by weight of dimethyl adipate, from 1% to 3% by weight of dimethyl succinate and from 1% to 3% by weight of dodecane; and relates to the non-therapeutic use of said composition for administration, in particular by spraying using a mister, in particular topical administration, in particular cutaneous administration to rabbits, in particular on the skin of a rabbit, or administration on an element of the environment such as objects and/or walls, in particular to promote well-being in rabbits, for example to promote positive emotional states in rabbits, in particular to facilitate adaptation to transport, during examination by the vet, during encounters with unfamiliar animals or people, during arrival in an unfamiliar place, or any other circumstance in the daily life of a rabbit.

In a particular embodiment according to the invention, the composition comprises a semiochemical composition, in particular comprising a soothing pheromone for a human, comprising from 25% to 35% by weight of methyl oleate, from 25% to 35% by weight of methyl palmitate, from 25% to 35% by weight of methyl linoleate, from 3% to 7% by weight of methyl stearate, from 3% to 5% by weight of methyl myristate and from 0.5% to 2% by weight of methyl pentadecanoate; and relates to the non-therapeutic use of said composition for administration, in particular by spraying using a mister, in particular topical administration, in particular cutaneous administration to a human, in particular on the skin of a human, or administration on an element of the environment such as textiles and/or objects and/or walls, in particular to promote well-being in humans.

In a particular embodiment according to the invention, the composition comprises a semiochemical composition, in particular comprising a sheep soothing pheromone, comprising from 30% to 40% by weight of methyl oleate, from 20% to 26% by weight of methyl linoleate, from 20% to 26% by weight of methyl palmitate, from 6% to 10% by weight of squalene, from 2% to 6% by weight of methyl myristate and from 3% to 5% by weight of methyl pentadecanoate; and relates to the non-therapeutic use of said composition for administration, in particular by spraying using a mister, in particular topical administration, in particular cutaneous administration to sheep or groups of sheep, in particular on the skin of a sheep or a group of sheep, in particular to promote soothing states in sheep.

In a particular embodiment according to the invention, the composition comprises a semiochemical composition, in particular comprising a feline facial pheromone, comprising from 62% to 70% by weight of methyl oleate, from 12% to 16% by weight of methyl palmitate, from 7% to 13% by weight of dimethyl pimelate and from 6% to 11% by weight of dimethyl azelate; and relates to the non-therapeutic use of said composition for administration, in particular by spraying using a mister, in particular topical administration, in particular cutaneous, to cats, in particular on the skin of a cat, or administration on an element of the environment such as textiles and/or objects and/or walls, in particular to prevent and manage stress-induced urinary marking behaviour in cats.

In a particular embodiment according to the invention, the composition comprises a semiochemical composition, in particular comprising a mixture of feline facial pheromone and cat soothing pheromone, comprising from 2% to 6% by weight of methyl laurate, from 2% to 6% by weight of methyl myristate, from 11% to 15% by weight of methyl palmitate, from 2% to 6% by weight of methyl stearate, from 42% to 50% by weight of methyl oleate, from 4% to 8% by weight of methyl linoleate, from 7% to 13% by weight of dimethyl pimelate and from 6% to 11% by weight of dimethyl azelate; and relates to the non-therapeutic use of said composition for administration, in particular by spraying using a mister, in particular topical administration, in particular cutaneous, to cats, in particular on the skin of a cat, or administration on an element of the environment such as objects and/or walls, in particular to prevent and manage stress-induced urinary marking behaviour in cats.

In a particular embodiment according to the invention, the composition comprises a semiochemical composition comprising from 40% to 60% by weight of 3-cyclopentylpropionic acid and from 40% to 60% by weight of 3-cyclohexylpropionic acid; and relates to the non-therapeutic use of said composition for administration, in particular by spraying using a mister, in particular topical administration, in particular cutaneous, to an animal, in particular a mammal, or to a human, or to animals treated as a group, or administration to an element of the environment such as textiles and/or objects and/or walls, in particular to prevent bites by haematophagous invertebrates, in particular haematophagous insects, such as mosquitoes, mites or ticks.

In a particular embodiment according to the invention, the composition comprises a semiochemical composition, in particular comprising an allomone produced by the uropygial glands of the duck, comprising from 40% to 60% by weight of bis(2-ethylhexyl) adipate and from 40% to 60% by weight of 1-isopropyl-2,2-dimethyltrimethylene diisobutyrate; and relates to the non-therapeutic use of said composition for administration, in particular by spraying using a mister, in particular topical administration, in particular cutaneous, to poultry such as hens, in particular on the skin of a poultry such as a hen, in particular as a repellent allomone, in particular to prevent infestation by parasites such as the poultry red mite (Dermanyssus sp. or Ornithonyssus sylviarum).

Another non-therapeutic use of said composition involves administration, in particular by spraying using a mist sprayer, in particular topical administration, in particular cutaneous, to an animal, for example birds or mammals, in particular on the skin of birds or mammals, or to a human, in particular on human skin, or administration to an element of the environment such as textiles and/or objects and/or walls, or by spraying into the air, in particular close to the nose of an animal or a human, in particular to prevent infestation or bites by haematophagous invertebrates, in particular haematophagous insects, such as mosquitoes, mites or ticks.

In a particular embodiment according to the invention, the composition comprises a semiochemical composition, in particular an equine soothing pheromone, comprising from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate; and relates to the non-therapeutic use of said composition for administration, in particular by spraying using a mister, in particular topical administration, in particular cutaneous, to horses, in particular on the skin of a horse, or administration on an element of the environment such as textiles and/or objects and/or walls, in particular to prevent social stress in horses and/or aggressive behaviour induced by encounters with unknown conspecifics, unknown people or animals of other species.

The above-mentioned uses of said composition according to the invention are non-therapeutic uses, and exclude any therapeutic use practised on the animal or human body.

### EXAMPLES

The following examples are given by way of illustration and therefore should not be construed to limit the scope of the invention.

### Example 1. Oil-In-Water Emulsions (Mist)

Compositions in the form of an oil-in-water emulsion (mist) according to the invention having the composition shown in the following Table 1 have been prepared.

TABLE 1

| | | | | Percentage |
|---|---|---|---|---|
| Compound name | Function | INCI | CAS No. | by weight |
| Aqueous phase | | | | |
| Water | | AQUA | 7732-18-5 | qsp |
| Glycerol | Humectant | Glycerin | 56-81-5 | 5% |
| Oily phase | | | | |
| Cremophor GS 32 | Emulsifying | POLYGLYCERYL-3 | 9009-32-9/ | 2.0% |
| | agent | DISTEARATE | 94423-19-5 | |
| Eumulgin CO 40 | Emulsifying | PEG-40 | 61788-85-0 | 2.0% |
| | agent | HYDROGENATED | | |
| | | CASTOR OIL | | |
| Polysorbate 80 or 20 | Emulsifying | POLYSORBATE 80 | 9005-65-6 or | 2.0% |
| | agent | | 9005-64-5 | |
| Myritol 318 | Emollient | CAPRYLIC/CAPRIC | 73398-61-5 | 2.0% |
| | agent | TRIGLYCERIDE | | |
| Aromastat | Antimicrobial | GLYCERIN/PHENETHYL | 56-81-5 | 1.0% |
| | agent | ALCOHOL | 60-12-8 | |
| | | CAPRYLHYDROXAMIC | 7377-03-9 | |
| | | ACID | | |
| Tinogard TT | Antioxidant | PENTAERYTHRITYL | 6683-19-8 | 0.1% |
| | agent | TETRA-DI-T-BUTYL | | |
| | | HYDROXYHYDROCINNAMATE | | |
| Fragrance (optional) | Additive | / | / | 1.0% |
| Semiochemical | | / | / | 1.98% |
| composition (SC1, | | | | |
| SC2, SC3, SC4 or | | | | |
| SC5, SC6, SC7, SC8, | | | | |
| SC9, SC10 or SC11) | | | | |
| 10% NaOH solution | | SODIUM HYDROXIDE | 1310-73-2 | 0.40% |
| (only with SC9) | | | | |

### Semiochemical Compositions (SC) Used as Examples in the Formulation of Table 1

SC1:27.40% by weight of methyl oleate, 22.70% by weight of methyl linoleate, 21.20% by weight of methyl behenate, 21.20% by weight of methyl palmitate, 4.50% by weight of methyl myristate and 3% by weight of methyl laurate; or
SC2: 35% by weight of methyl linoleate, 30% by weight of methyl oleate, 20% by weight of methyl palmitate, 7% by weight of methyl myristate, 6% by weight of methyl laurate and 2% by weight of methyl decanoate; or
SC3: 29% by weight of methyl linoleate, 29% by weight of methyl oleate, 18% by weight of methyl palmitate, 13% by weight of methyl stearate, 5% by weight of methyl myristate, 4% by weight of methyl pentadecanoate and 2% by weight of methyl laurate; or
SC4: 35% by weight of methyl linoleate, 29% by weight of methyl oleate, 18% by weight of methyl palmitate, 9.18% by weight of 2-(2-butoxyethoxy) ethanol, 4.32% by weight of dimethyl glutarate, 1.62% by weight of dimethyl adipate, 1.62% by weight of dimethyl succinate and 1.26% by weight of dodecane; or
SC5:30.80% by weight of methyl oleate, 30% by weight of methyl palmitate, 28.80% by weight of methyl linoleate, 5.50% by weight of methyl stearate, 3.55% by weight of methyl myristate and 1.35% by weight of methyl pentadecanoate; or
SC6:35.3% by weight of methyl oleate, 23.7% by weight of methyl linoleate, 23.3% by weight of methyl palmitate, 8.6% by weight of squalene, 5.2% by weight of methyl myristate and 3.9% by weight of methyl pentadecanoate; or
SC7:67.2% by weight of methyl oleate, 13.60% by weight of methyl palmitate, 11.40% by weight of dimethyl pimelate and 7.80% by weight of dimethyl azelate; or
SC8:4.50% by weight of methyl laurate, 4.50% by weight of methyl myristate, 13.40% by weight of methyl palmitate, 4.50% by weight of methyl stearate, 46.6% by weight of methyl oleate, 69.50% by weight of methyl linoleate, 11% by weight of dimethyl pimelate and 9% by weight of dimethyl azelate; or
SC9: 50% by weight of 3-cyclopentyl propionic acid and 50% by weight of 3-cyclohexyl propionic acid; or
SC10: 50% by weight of bis(2-ethylhexyl) adipate and 50% by weight of 1-isopropyl-2,2-dimethyltrimethylene diisobutyrate; or
SC11: from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate.

### Example 2. Process for Preparing Oil-In-Water Emulsions (Mist) According to Example 1

The oil-in-water emulsions (mist) according to Example 1 were prepared according to the following protocol:

### Step 1: Preparing the Paste/Aqueous Phase

1. The glycerine was weighed and added to a container adapted to contain the final volume of the aqueous phase. The mixture was allowed to cool to room temperature.

2. Once the pasting was complete, all the water was added.

3. After homogenising the aqueous phase (obtaining a homogeneous mixture), the aqueous phase was kept at 70° C. and stirred until step 3.

### Step 2: Preparing the Oily Phase

4. The components of the oily phase, namely Cremophor GS 32, Eumulgin CO 40, Polysorbate 80 or Polysorbate 20, Myritol 318, Tinogard TT, Aromastat and the semiochemical composition (SC1, SC2, SC3, SC4 or SC5, SC6, SC7, SC8, SC9 or SC10) were weighed and added into another container adapted to contain the final volume of the oily phase.

5. Once all the compounds were added, the mixture was stirred and heated to 70° C. until a homogeneous mixture was obtained.

### Step 3: Formation of the Oil-In-Water Emulsion

6. When the two phases (aqueous and oily) were perfectly homogeneous, the oily phase was added at 70° C. to the aqueous phase at 70° C. Care was taken to ensure that the emulsion was well agitated throughout this stage. The emulsion was then cooled to room temperature after homogenising the emulsion.

Step 4: Modification of pH Only with the Above-Mentioned SC9 Composition

7. In the case of the composition comprising the semiochemical composition SC9, and in order to increase the pH, a pH adjustment step was carried out by adding a 10% NaOH solution until a pH of between 6 and 7 was obtained.

### Step 5: Perfume

8. Add any perfume with stirring at room temperature

### Step 6: Recovery of the Oil-In-Water (O/W) Emulsion

The final emulsion obtained was fluid, opalescent/transparent and of low viscosity.

### Example 3. Organoleptic Measurements of Oil-In-Water Emulsions (Mist) According to Example 1

3 criteria (appearance, colour, odour) were selected for organoleptic measurements.
Appearance: Fluid liquid
Colour: White or opalescent/transparent/translucent
Odour: Characteristic odour.

### Example 4. PH Measurements of Oil-In-Water Emulsions (Mist) According to Example 1

The pH measurements were carried out using a LE 438 IP 67 electrode marketed by Mettler Toledo®. This is a 2-in-1 glass pH electrode with an integrated temperature sensor and liquid electrolyte for rapid measurements in the laboratory; for aqueous samples such as acids, bases, etc.

The specifications for this electrode are given in Table 2 below.

TABLE 2

| | |
|---|---|
| Measuring range | pH 0-14 |
| Zero point | 7.00 ± 0.25 |
| Temperature range | 0-80° C. |
| Temperature sensor | NTC 30 K Ω |
| Membrane resistance (25° C.) | <250 MΩ |
| Reference electrolyte | 3 mol/L KCl solution |
| | saturated with AgCl |
| Reference system | Ag/AgCl |
| Reference diaphragm | Gel |

Before any measurements are taken, the pH meter used is calibrated. The nature of the semiochemical composition can affect the pH of the emulsion, as some semiochemicals have an acid pH. For topical application, particularly to the skin, it is therefore necessary to measure the pH of each emulsion obtained and, if necessary, adjust the pH to obtain a pH of between 5 and 8, preferably a pH of between 5.5 and 7.5, or a pH of between 6.0 and 7.5.

pH measurements were carried out after each emulsion was obtained and are reported below:
MIST with SC1: 5.0<pH<7.5
MIST with SC2: 5.0<pH<7.5
MIST with SC3: 5.5<pH<7.5
MIST with SC4:5.5<pH<7.5
MIST with SC5: 5.5<pH<7.5
MIST with SC6: 5.5<pH<7.5
MIST with SC7: 5.5<pH<7.5
MIST with SC8: 5.5<pH<7.5
MIST with SC10: 5.5<pH<7.5
MIST with SC11: 5.5<pH<7.5
MIST with SC9: 3.5<pH<4.5.

In order to get as close as possible to the pH of animal or human skin, and to avoid causing an irritation reaction, the pH of the formulation corresponding to MIST with SC9 was adjusted. Following this adjustment, new pH measurements were carried out and are reported below: MIST with SC9: 6.0<pH<7.5.

For formulations corresponding to MIST with SC1, SC2, SC3, SC4, SC5, SC6, SC7, SC8, SC10 and SC11, no pH adjustment step was carried out.

### Example 5. Measuring the Viscosity of Oil-In-Water Emulsions (Mist) According to Example 1

Viscosity is the resistance of a fluid to sliding from one layer to another.

For each formulation, the viscosity must be checked after each production run. Viscosity is influenced by a number of factors, including temperature, the measurement device used, the measurement speed used and the time of measurement during production (measurements at T0 and T+24 h, T+48 h). The viscosity should be measured when the solution is at room temperature, preferably at T+24 h.

The viscosity of the oil-in-water emulsions according to Example 1 was measured using a Brookfield digital viscometer (model DV-I Prime), mobile S 61 (large mobile part) and at a rotation speed of 0.6 RPM.

The Brookfield DV-I Prime viscometer measures the viscosity of fluids at a given velocity gradient. The principle of operation involves rotating a moving part (which is immersed in the liquid to be tested) by means of a calibrated spring. The measurement range (in centipoises or milliPascal-seconds) is determined by the speed of rotation of the device, the size and shape of the device, the cross-section where the device is rotating and the full scale of the torque of the calibrated spring.

The specifications of the Brookfield DV-I Prime viscometer are given in Table 3 below.

TABLE 3

| | |
|---|---|
| Speeds (rpm) | 0.0; 0.3; 0.6; 1.5; 3; 6; |
| | 12; 30; 60; 0.5; 1; 2; |
| | 2.5; 4; 5; 10; 20; 50; 100 |
| Viscosity range (cP) | 15-2,000,000 |
| Operating medium | Temperature range 0° C. |
| | to 40° C. (32° F. to |
| | 104° F.) 20% to 80% |
| | R.H. non-condensing |
| Analogue torque output | 0-1 Vdc (torque from 0 to 100%) |
| Accuracy of viscosity | ±1° C:-full scale |
| measurement | |
| Reproducibility of | 0.2% of full scale used |
| viscosity measurement | |
| Devices used | S61; S62; S63; S64 |

Following these measurements, all the oil-in-water emulsions (mist) according to Example 1 had a viscosity of between 1 and 50 cP or mPa·s, with the exception of the emulsion comprising the semiochemical composition SC9, which had a viscosity of between 3500 and 8000 cP or mPa·s. Viscosity was notably lower in the presence of a perfume or a NaOH solution (the percentage of water being higher).

### Example 6. Test to Identify the Presence or Absence of Stains Following Spraying of the Semiochemical Composition SC9 on Textiles of Different Materials

### Protocol

Place the garment on a hanger, take a photo before application (T0)
Spray the semiochemical composition SC9 20-30 cm from the garment on different areas [10 sprays per garment, if the garment is in 2 parts (e.g. trousers) spray 5 times per side]
Photo after application (T1)
Take a photo 1 hour after application (T2)
Wash the garments in a washing machine (standard programme at 30° C.)
Take a photo after washing (T3).

### Material

### Solution Used

### Semiochemical Composition SC9

Garments and their Composition

The compositions and types of garments used are shown in Table 4 below.

TABLE 4

| Composition | Type of garment |
|---|---|
| 100% cotton | White polo shirt |
| | Blue T-shirt |
| 100% polyester | Blue T-shirt |
| | Black polo shirt |
| 35% polyamide, 30% wool, | Blue jumper |
| 30% viscose, | |
| 5% cashmere | |
| 100% wool | Cardigan |
| 60% recycled wool, | Trousers |
| 35% polyester, 5% | |
| polyamide | |
| 100% silk | Tie |
| 100% synthetic | Black dress |

### Results

The results of the tests carried out by the inventors are reported in Table 5 below.

TABLE 5

| Type of garment | Material | T0 | T1 | T2 | T3 |
|---|---|---|---|---|---|
| White polo shirt | 100% cotton | CLEAR | CLEAR | CLEAR | CLEAR |
| Blue T-shirt | | Water | CLEAR | CLEAR | CLEAR |
| | | mark | | | |
| Blue T-shirt | 100% polyester | CLEAR | CLEAR | CLEAR | CLEAR |
| Black polo | | Droplets | CLEAR | CLEAR | CLEAR |
| shirt | | deposited | | | |
| | | on fibres | | | |
| Blue jumper | 35% polyamide, | CLEAR | CLEAR | CLEAR | CLEAR |
| | 30% wool, 30% | | | | |
| | viscose, 5% | | | | |
| | cashmere | | | | |
| Cardigan | 100% wool | CLEAR | CLEAR | CLEAR | CLEAR |
| | | | | | (shrunk) |
| Trousers | 60% recycled | Droplets | CLEAR | CLEAR | CLEAR |
| | wool, 35% | of water | | | |
| | polyester, 5% | settle on | | | |
| | polyamide | the fibres | | | |
| Tie | 100% silk | Water | CLEAR | CLEAR | CLEAR |
| | | mark | | | |
| Black dress | Synthetic | Droplets | CLEAR | CLEAR | CLEAR |
| | | deposited | | | |
| | | on fibres | | | |

CLEAR = Nothing to report, i.e. no change observed.

The above-mentioned results showed that no stains were observed on textiles of different materials following spraying with the semiochemical composition SC9.

### Example 7. Acceptability Tests on Cats and Rabbits

The results of acceptability tests in cats carried out by the inventors are reported in Table 6 below.

TABLE 6
Results of acceptability questionnaire - Chat

| Reaction | High-pitched cries, | Ears on the | The cat remains in | The cat tries to | The cat stays | The cat shows |
|---|---|---|---|---|---|---|
| DURING | without flight or | back of the | place, but its tail is | get out of the | in place, but | no particular |
| application | defensive | head, hissing, | wagging | way without | with its ears | reaction |
| | movements | active | | reacting abruptly | pinned to the | |
| | | defensive | | during the | back of its | |
| | | reactions | | application | head or in a | |
| | | | | | police hat | |
| | | | | | position | |
| Number of | 0 | 0 | 0 | 4 | 0 | 9 |
| reactions | | | | | | |
| observed | | | | | | |
| Reaction AFTER | The cat hides and | The cat stays put, but can no longer | | The cat shows a very active and | | After a short |
| APPLICATION | is unapproachable | be touched (threatening mimics and | | long grooming behaviour | | period of |
| | (threatening mimics | vocalizations) | | | | licking, the cat |
| | and vocalisations) | | | | | resumes its |
| | | | | | | usual activities |
| Number of | 0 | 0 | | 3 | | 10 |
| reactions | | | | | | |
| observed | | | | | | |
| Average product | 7/10 | | | | | |
| odour analogue | | | | | | |
| scale | | | | | | |

The results of acceptability tests in rabbits carried out by the inventors are reported in Table 7 below.

TABLE 7
Results of acceptability questionnaire-Rabbit

| | Sudden flight | Defence | | | No |
|---|---|---|---|---|---|
| Reaction | movements | movements | | The rabbit | reaction |
| DURING | (jumping, forward | with | | lies flat | or shakes |
| application | movement) | the hind legs | Screams | on the table | slightly |
| Number of | 0 | 0 | 0 | 0 | 4 |
| reactions | | | | | |
| observed | | | | | |
| Reaction AFTER | The rabbit won't | The rabbit | Rabbits groom for | The rabbit shows | |
| APPLICATION | let anyone near it | remains pinned to | long periods of | no reaction and | |
| | (escape) | the ground, | time | resumes its usual | |
| | | trembling | | activities | |
| Number of | 0 | 0 | 1 | 3 | |
| reactions | | | | | |
| observed | | | | | |
| Average product | 7/10 | | | | |
| odour analogue | | | | | |
| scale | | | | | |

### REFERENCES

Silverstein, R. M., 1977. “Complexity, diversity and specificity of behaviour-modifying chemicals; examples from Coleoptera and Hymenoptera,” Chemical Control of Insect Behavior: Theory and Application. Publishers H. H. Shorey & J. J. Mckelvey pp. 231-251 New York: Plenum Press);
Wyatt T. D., 2003 “Pheromones and Animal Behavior. Communication by smell and taste”, pp. 1-4 New York: Cambridge University Press.

1. A composition comprising:
a) an aqueous phase comprising water and at least one humectant chosen from polyols and sugars,
b) an oily phase comprising at least one emollient agent, at least one emulsifying agent, and a semiochemical composition,
wherein the semiochemical composition comprises semiochemical compounds selected from the group consisting of:
(i) a mixture comprising methyl oleate and methyl palmitate;
(ii) a mixture of 3-cyclopentyl propionic acid and 3-cyclohexyl propionic acid; and
(iii) a mixture of bis(2-ethylhexyl) adipate and 1-isopropyl-2,2-dimethyltrimethylene diisobutyrate;
wherein the composition is in the form of an oil-in-water (O/W) emulsion.

2. The composition according to claim 1, wherein the mixture comprising methyl oleate and methyl palmitate is a mixture comprising methyl linoleate, methyl oleate and methyl palmitate.

3. The composition according to claim 1, wherein the semiochemical composition comprises semiochemical compounds selected from the group consisting of:
(a) a mixture of methyl oleate, methyl linoleate, methyl behenate, methyl palmitate, methyl myristate and methyl laurate;
(b) a mixture of methyl linoleate, methyl oleate, methyl palmitate, methyl myristate, methyl laurate and methyl decanoate;
(c) a mixture of methyl linoleate, methyl oleate, methyl palmitate, methyl stearate, methyl myristate, methyl pentadecanoate and methyl laurate;
(d) a mixture of methyl linoleate, methyl oleate, methyl palmitate, 2-(2-butoxyethoxy) ethanol, dimethyl glutarate, dimethyl adipate, dimethyl succinate and dodecane;
(e) a mixture of methyl oleate, methyl palmitate, methyl linoleate, methyl stearate, methyl myristate and methyl pentadecanoate;
(f) a mixture of methyl oleate, methyl linoleate, methyl palmitate, squalene, methyl myristate and methyl pentadecanoate;
(g) a mixture of methyl oleate, methyl palmitate, dimethyl pimelate and dimethyl azelate;
(h) a mixture of methyl laurate, methyl myristate, methyl palmitate, methyl stearate, methyl oleate, methyl linoleate, dimethyl pimelate and dimethyl azelate;
(i) a mixture of methyl oleate, methyl palmitate, methyl linoleate, dioxolane, methyl laurate and methyl myristate;
(j) a mixture of 3-cyclopentyl propionic acid and 3-cyclohexyl propionic acid; and
(k) a mixture of bis(2-ethylhexyl) adipate and 1-isopropyl-2,2-dimethyltrimethylene diisobutyrate.

4. The composition according to claim 1, wherein the semiochemical composition comprises:
(a) from 25% to 35% by weight of methyl oleate, from 20% to 25% by weight of methyl linoleate, from 20% to 25% by weight of methyl behenate, from 20% to 25% by weight of methyl palmitate, from 3% to 6% by weight of methyl myristate and from 2% to 4% by weight of methyl laurate; or
(b) from 30% to 40% by weight of methyl linoleate, from 25% to 35% by weight of methyl oleate, from 15% to 25% by weight of methyl palmitate, from 5% to 9% by weight of methyl myristate, from 5% to 7% by weight of methyl laurate and from 1% to 3% by weight of methyl decanoate; or
(c) from 25% to 35% by weight of methyl linoleate, from 25% to 35% by weight of methyl oleate, from 15% to 25% by weight of methyl palmitate, from 10% to 15% by weight of methyl stearate, from 2% to 7% by weight of methyl myristate, from 2% to 6% by weight of methyl pentadecanoate and from 1% to 3% by weight of methyl laurate; or
(d) from 30% to 40% by weight of methyl linoleate, from 25% to 35% by weight of methyl oleate, from 15% to 25% by weight of methyl palmitate, from 5% to 15% by weight of 2-(2-butoxyethoxy) ethanol, from 2% to 6% by weight of dimethyl glutarate, from 1% to 3% by weight of dimethyl adipate, from 1% to 3% by weight of dimethyl succinate and from 1% to 3% by weight of dodecane; or
(e) from 25% to 35% by weight of methyl oleate, from 25% to 35% by weight of methyl palmitate, from 25% to 35% by weight of methyl linoleate, from 3% to 7% by weight of methyl stearate, from 3% to 5% by weight of methyl myristate and from 0.5% to 2% by weight of methyl pentadecanoate; or
(f) from 30% to 40% by weight of methyl oleate, from 20% to 26% by weight of methyl linoleate, from 20% to 26% by weight of methyl palmitate, from 6% to 10% by weight of squalene, from 2% to 6% by weight of methyl myristate and from 3% to 5% by weight of methyl pentadecanoate; or
(g) from 62% to 70% by weight of methyl oleate, from 12% to 16% by weight of methyl palmitate, from 7% to 13% by weight of dimethyl pimelate and from 6% to 11% by weight of dimethyl azelate; or
(h) from 2% to 6% by weight of methyl laurate, from 2% to 6% by weight of methyl myristate, from 11% to 15% by weight of methyl palmitate, from 2% to 6% by weight of methyl stearate, from 42% to 50% by weight of methyl oleate, from 4% to 8% by weight of methyl linoleate, from 7% to 13% by weight of dimethyl pimelate and from 6% to 11% by weight of dimethyl azelate; or
(i) from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate; or
(j) from 40% to 60% by weight of 3-cyclopentyl propionic acid and from 40% to 60% by weight of 3-cyclohexyl propionic acid; or
(k) from 40% to 60% by weight of bis(2-ethylhexyl) adipate and from 40% to 60% by weight of 1-isopropyl-2,2-dimethyltrimethylene diisobutyrate;
the total sum of the semiochemical compounds present in the semiochemical composition being equal to 100%.

5. The composition according to claim 1, wherein the at least one humectant is chosen from polyols selected from the group consisting of glycerol, propylene glycol, polyethylene glycols (PEG), xylitol, isomalt, maltitol, mannitol, polydextrose, sorbitol, isomaltitol, lactitol, and mixtures thereof; and/or from sugars selected from the group consisting of fructose, glucose and lactose.

6. The composition according to claim 5, wherein the at least one humectant is glycerol.

7. The composition according to claim 1, wherein the at least one humectant is present in an amount ranging from 4% to 12% by weight, relative to the total weight of the composition.

8. The composition according to claim 1, which comprises from 60% to 95% by weight of water, relative to the total weight of the composition, and/or from 1% to 4% by weight of the semiochemical composition, relative to the total weight of the composition.

9. The composition according to claim 1, wherein the at least one emollient agent is selected from the group consisting of hydrogenated vegetable oils, silicone oils, mineral oils, waxes, fatty alcohols, fatty acids, fatty acid esters, glyceride derivatives and glycerol derivatives.

10. The composition according to claim 9, wherein the at least one emollient agent is a glyceride derivative.

11. The composition according to claim 1, wherein the at least one emollient agent is present in an amount ranging from 0.5% to 8% by weight, relative to the total weight of the composition.

12. The composition according to claim 1, wherein the at least one emulsifying agent is selected from the group consisting of polyoxyethylene sorbitan fatty acid esters, polyol fatty acid esters, and hydrogenated and ethoxylated vegetable oils.

13. The composition according to claim 1, wherein the at least one emulsifying agent is present in an amount ranging from 1% to 25% by weight, relative to the total weight of the composition.

14. The composition according to claim 1, wherein the oily phase further comprises at least one additive selected from the group consisting of a preservative, a perfume, a masking agent, and mixtures thereof.

15. The composition according to claim 1, which is administered in the form of a mist.

16. The composition according to claim 15, which is administered in the form of a mist using a mister.

17. The composition according to claim 1, wherein it comprises:
a) an aqueous phase comprising water and glycerol,
b) an oily phase comprising polyglyceryl-3 distearate, PEG-40 hydrogenated castor oil, polyoxyethylene sorbitan fatty acid esters, triglycerides of caprylic acid and capric acid, glycerine/phenyl alcohol/caprylhydroxamic acid, pentaerythrityl tetra-di-t-butyl hydroxyhydrocinnamate, an optional perfume, and a semiochemical composition,
the semiochemical composition being present in the composition in the form of an O/W emulsion in an amount ranging from 1% to 4% by weight, relative to the total weight of the composition.

18. A process for preparing the composition in the form of an oil-in-water (O/W) emulsion according to claim 1, comprising the following steps:
a) the at least one gelling agent is pasted into the at least one humectant; then water is added with stirring and at a temperature of between 65° C. and 75° C., so as to obtain a homogeneous aqueous phase;
b) the at least one emollient agent, the at least one emulsifying agent, the semiochemical composition and optionally the at least one additive are mixed, with stirring and at a temperature of between 65° C. and 75° C., so as to obtain a homogeneous oily phase;
c) the oily phase from step b) is added, with stirring, to the aqueous phase from step a) so as to obtain an O/W emulsion;
d) optionally, the pH of the emulsion obtained in step c) is adjusted;
e) optionally, a perfume is added;
f) the composition is recovered in the form of an O/W emulsion obtained in step c), d) or e).

19. The process according to claim 18, wherein:
the pasting step a) is carried out at room temperature; and/or
the homogeneous aqueous phase obtained in step a) is kept under stirring at a temperature of between 65° C. and 75° C.; and/or
the homogeneous oily phase obtained in step b) is kept under stirring at a temperature of between 65° C. and 75° C.; and/or
step c) is carried out by mixing the oily phase from step b) with the aqueous phase from step a) at a temperature of between 65° C. and 75° C., and the emulsion is cooled to room temperature; and/or
step d) is carried out by adding a basic solution.

20. Non-therapeutic use of the composition in the form of an oil-in-water (O/W) emulsion according to claim 1, wherein the use comprises administration of the composition according to claim 1 on the skin of an animal or a human, or on the skin of farm animals treated as a group, or on textiles and/or objects and/or walls, or by spraying into the air.

21. The non-therapeutic use according to claim 20, wherein the composition is administered by spraying with the aid of a mister.

22. The non-therapeutic use according to claim 20, wherein the composition is sprayed into the air, close to the nose of an animal or a human.
